(19) United States
(12) Patent Application Publication
(10) Pub. No.: US 2024/0041745 A1
(43) Pub. Date: Feb. 8, 2024

(54) COMPOSITION FOR CARING FOR THE KERATIN MATERIALS

(71) Applicant: L'OREAL, Paris (FR)

(72) Inventors: Xiaomeng WU, Shanghai (CN); Xiaomin WENG, Shanghai (CN)

(73) Assignee: L'OREAL, Paris (FR)

(21) Appl. No.: 18/258,777

(22) PCT Filed: Dec. 22, 2020
(86) PCT No.: PCT/CN2020/138260

§ 371 Date: Jun. 21, 2023

(51) Int. Cl. A61K 8/67; A61K 8/03; A61K 8/02; A61Q 19/00

(52) U.S. Cl. CPC A61K 8/671; A61K 8/03; A61K 8/67; A61K 8/673; A61K 8/025; A61K 8/675; A61Q 19/00

(57) ABSTRACT

Provided herein is a bi-phase composition comprising: (A) a continuous aqueous phase; (B) an oil phase comprising an unstable active agent, and (C) hydrophobic particles adhered on the surface of the oil phase.

### TECHNICAL FIELD

The present invention relates to a composition caring for keratin materials, and in particular the skin. Further, the present invention relates to use of same for caring for keratin materials, especially the skin.

### BACKGROUND ART

It has always been an ultimate goal of the cosmetic filed to deliver products with skin benefits such as hydration, moisturizing, whitening, cleansing, and so on.

Some oil soluble/dispersible active agents are generally less stable in solvated/dispersed state, e.g., in aqueous solution, compared with the dry form thereof, thus impacting the cosmetic effect thereof. For example, some oil soluble vitamins, such as retinoids, e.g., retinol (also known as vitamin A), is more readily to be reacted in aqueous phase.

It is known, in particular in food and pharmaceutical field, to formulate these types of ingredients in dry form, such as beads, granules, particles, tablets, etc., or in a highly-sticky form, such as cream, rich lotion, etc.

For example, traditional cosmetic compositions with high retinol content generally are prepared in cream or rich lotion, because such systems are usually easy to improve retinol stability in formula. On the other hand, however, such formulas are very rich and oily to skin. Meanwhile, compared with light and fresh texture of cosmetic water, the cream and rich lotion also show poor performance in penetration efficiency, which means more retinol should be added in to formula to meet the beauty requirements, that may lead to potential irritation problems.

Based on the foregoing, there is a need for a new type of composition caring for the keratin materials, in particular the skin.

### SUMMARY OF THE INVENTION

Therefore, in one aspect, it is preferable to provide a composition for caring for the keratin materials to dispense oil soluble/dispersible active agents more effectively, in particular for a composition comprising a solvent.

In another aspect, it is preferable to provide a composition for caring for the keratin materials, in particular the skin, with an improved easy usage.

One subject of the invention is thus to provide a bi-phase composition comprising:
(A) a continuous aqueous phase;
(B) an oil phase comprising an unstable active agent, and
(C) hydrophobic particles adhered on the surface of the oil phase.

The composition according to the present invention may preferably comprise an antioxidant, either in the aqueous phase (A) and/or in the oil phase (B).

Another subject of the present invention is to provide a package comprising:
1) a container; and
2) the bi-phase composition according to the present invention;
wherein the aqueous phase (A) and the oil phase (B) is placed in such a way that when the container is opened, the oil phase (B) is separate from environment by the aqueous phase (A).

Another subject of the present invention is use of the composition according to the present invention for caring for the keratin materials, especially the skin. This use may manifest itself as a process for caring for the keratin materials, especially the skin.

Other characteristics and advantages of the present invention will emerge more clearly on reading the description and the examples that follow.

### DETAILED DESCRIPTION OF INVENTION

Throughout the description, including the claims, the term “comprising a” should be understood as being synonymous with “comprising at least one”, unless otherwise mentioned. Moreover, the expression “at least one” used in the present description is equivalent to the expression “one or more”.

Preferably, the “keratin material” according to the present invention is the skin. By “skin”, we intend all the body skin. Still preferably, the keratin material is the face, or the neck, especially the face.

In the application, unless specifically mentioned otherwise, contents, parts and percentages are expressed on a weight basis.

The present invention provides a bi-phase composition comprising:
(A) a continuous aqueous phase;
(B) an oil phase comprising an unstable active agent, and
(C) hydrophobic particles adhered on the surface of the oil phase.

The composition according to the present invention may preferably comprise an antioxidant, either in the aqueous phase (A) and/or in the oil phase (B).

The present invention also provides a package comprising:
1) a container; and
2) the bi-phase composition according to the present invention;
wherein the aqueous phase (A) and the oil phase (B) is placed in such a way that when the container is opened, the oil phase (B) is separate from environment by the aqueous phase (A).

Aqueous Phase (A)

The composition according to the present invention comprises an aqueous phase.

The aqueous phase comprises water.

The aqueous phase may also comprise water-miscible organic solvents (at room temperature: 20-25° C.), for instance monoalcohols containing from 2 to 6 carbon atoms, such as ethanol or isopropanol; polyols especially containing from 2 to 20 carbon atoms, preferably containing from 2 to 10 carbon atoms and preferentially containing from 2 to 6 carbon atoms, such as glycerol, propylene glycol, butylene glycol, pentylene glycol, hexylene glycol, dipropylene glycol or diethylene glycol; glycol ethers (especially containing from 3 to 16 carbon atoms) such as mono-, di- or tripropylene glycol (C₁-C₄)alkyl ethers, mono-, di- or triethylene glycol (C₁-C₄)alkyl ethers, and mixtures thereof.

The aqueous phase may also comprise any water-soluble or water-dispersible compound that is compatible with an aqueous phase, such as hydrophilic gelling agents, preservatives or surfactants, and mixtures thereof.

In particular, the composition of the present invention may comprise the aqueous phase in an amount ranging from 65% to 99% by weight, especially from 70% to 90% by weight and more particularly from 75% to 85% by weight, relative to the total weight of the composition.

Polysaccharide

The composition according to the invention, in particular for oil phase suspending in water phase system, can comprise at least one polysaccharide, in particular in the aqueous phase, which is preferably of biotechnological origin.

In particular, these polysaccharides may, where appropriate, be chemically modified to promote its hydrophilic valency.

As examples of polysaccharides that may be used according to the invention, mention may be made especially of:
a) algal extracts such as alginates, carrageenans and agar-agar, and mixtures thereof. Examples of carrageenans that may be mentioned include Satiagum UTC30® and UTC10® from the company Degussa; an alginate that may be mentioned is the sodium alginate sold under the name Kelcosol® by the company ISP;
b) gums, such as xanthan gum, gellan gum, guar gum and nonionic derivatives thereof (hydroxypropyl guar), gum arabic, konjac gum or mannan gum, gum tragacanth, ghatti gum, karaya gum, locust bean gum, agar gum, scleroglucan gums and mixtures thereof; examples that may be mentioned include the xanthan gum sold under the name Keltrol® CG-T by the company CP Kelco, gellan gum sold under the name Kelcogel® CG LA by the company CP Kelco, guar gum sold under the name Jaguar HP 105® by the company Rhodia; mannan gum and konjac Gum® (1% glucomannan) sold by the company GfN;
c) starches, which are preferably modified, such as those derived, for example, from cereals such as wheat, corn or rice, from legumes such as white lentil, from tubers such as potato or cassava, tapioca starches; dextrins, such as corn dextrins; Amidon de Mais B® from the company Roquette; potato feculent modified with 2-chloroethylaminodipropionic acid neutralized with sodium hydroxide, sold under the name Structure Solanace® by the company National Starch; native tapioca starch powder sold under the name Tapioca Pure® by the company National Starch;
d) dextrins, such as dextrin extracted from corn under the name Index® from the company National Starch;
e) pectins,
f) chitosan and derivatives thereof,
g) polyholosides comprising at least two saccharides, preferably of natural origin, and especially chosen from:
   aldoses such as
   pentoses: ribose, arabinose, xylose or apiose, for example,
   hexoses: glucose, fucose, mannose or galactose, for example,
   ketoses such as fructose,
   deoxyoses, such as rhamnose, digitoxose, cymarose or oleandrose,
   saccharide derivatives such as uronic acids, for instance mannuronic acid, guluronic acid, galacturonic acid or glycuronic acid, or itols, for instance mannitol or sorbitol.

Mention may be made in particular of the polyholosides comprising fucose, galactose and galacturonic acid units, and for example a linear sequence of α-L-fucose, α-D-galactose and galacturonic acid, for instance the biosaccharide gum-1 sold under the trade name Fucogel® 1000 PP or Fucogel® 1.5P by the company Solabia,
h) anionic polysaccharides, in particular of biotechnological origin, such as anionic polysaccharide bearing as repeating unit a tetrasaccharide composed of L-fucose, D-glucose and glucuronic acid, such as the product bearing the INCI name Biosaccharide Gum-4 sold under the reference Glycofilm 1.5P by the company Solabia,
i) and mixtures thereof.

Preferably, the polysaccharide of the present invention is chosen from:
gums such as xanthan gum, or gellan gum;
polyholosides comprising fucose, galactose and galacturonic acid units, for example biosaccharide gum-1.

According to a preferred embodiment, the composition of the present invention comprises from 0.0001% to 5% by weight, preferably from 0.001% to 2% by weight, more preferably from 0.005% to 1% by weight of the polysaccharides, relative to the total weight of the composition.

Oil Phase (B)

The composition according to the invention can comprise an oil phase comprising a cosmetically acceptable fatty substance.

The term “fatty substance” means organic compounds that are insoluble in water at ordinary temperature (25° C.) and at atmospheric pressure (760 mmHg) (solubility of less than 5% by weight, preferably 1% by weight and even more preferentially 0.1% by weight). They may preferably have in their structure a sequence of at least two siloxane groups or at least one hydrocarbon-based chain comprising at least 6 carbon atoms. In addition, the fatty substances are generally soluble in organic solvents under the same temperature and pressure conditions, for instance chloroform, ethanol, benzene or decamethylcyclo pentasiloxane.

The fatty substances are especially chosen from lower alkanes, fatty alcohols, fatty acid esters, fatty alcohol esters, oils, in particular mineral, plant, animal or synthetic non-silicone oils, non-silicone waxes, and silicone oils.

It is recalled that, for the purposes of the invention, the fatty alcohols, fatty esters and fatty acids more particularly contain one or more linear or branched, saturated or unsaturated hydrocarbon-based groups comprising 6 to 30 carbon atoms, which is (are) optionally substituted, in particular with one or more hydroxyl groups (in particular 1 to 4). If they are unsaturated, these compounds may comprise one to three conjugated or unconjugated carbon-carbon double bonds.

As regards lower alkanes, these alkanes comprise from 6 to 16 carbon atoms and are linear or branched, optionally cyclic. By way of example, the alkanes may be chosen from hexane and dodecane, isoparaffins such as isohexadecane and isodecane.

Non-silicone oils that may be used in the composition of the invention can comprise particularly “hydrocarbon-based oil” (or “hydrocarbonated oil”, or “hydrocarbon oil”), which means an oil formed essentially from, or even constituted by, carbon and hydrogen atoms, and optionally oxygen and nitrogen atoms, and not containing any silicon or fluorine atoms.

Examples of the non-silicone oils that may be mentioned include:
hydrocarbon-based oils of animal origin, such as perhydrosqualene;
hydrocarbon-based oils of plant origin, such as liquid fatty acid triglycerides containing from 6 to 30 carbon atoms, for instance heptanoic or octanoic acid triglycerides, or alternatively, for example, sweet almond oil, sunflower oil, corn oil, soybean oil, marrow oil, grapeseed oil, sesameseed oil, hazelnut oil, apricot oil, macadamia oil, arara oil, castor oil, avocado oil, caprylic/capric acid triglycerides, for instance those sold by the company Stearineries Dubois or those sold under the names Miglyol® 810, 812 and 818 by the company Dynamit Nobel, jojoba oil and shea butter oil;
linear or branched hydrocarbons of more than 16 carbon atoms and of mineral or synthetic origin, such as liquid paraffins, petroleum jelly, liquid petroleum jelly, polydecenes, and hydrogenated polyisobutenes such as Parleam®;
fluoro oils, for instance perfluoromethylcyclo pentane and perfluoro-1,3-dimethylcyclohexane, sold under the names Flutec® PC1 and Flutec® PC3 by the company BNFL Fluorochemicals; perfluoro-1,2-dimethylcyclobutane; perfluoroalkanes such as dodecafluoropentane and tetradecafluorohexane, sold under the names PF 5050® and PF 5060® by the company 3M, or bromoperfluorooctyl sold under the name Foralkyl® by the company Atochem; nonafluoromethoxy butane and nonafluoroethoxyisobutane; perfluoromorpho line derivatives such as 4-trifluoromethyl perfluoromorpholine sold under the name PF 5052® by the company 3M.

The various oils, in particular hydrocarbon-based oil, are preferably present in the oil phase. Optionally, the oil phase can consist of, or consist essentially of, the oil, e.g., the hydrocarbon-based oil.

The fatty alcohols that may be used in the composition of the invention are not oxyalkylenated. They are saturated or unsaturated, linear or branched and comprise from 6 to 30 carbon atoms and more particularly from 8 to 30 carbon atoms. Mention may be made of cetyl alcohol, stearyl alcohol and the mixture thereof (cetylstearyl alcohol), octyldodecanol, 2-butyloctanol, 2-hexyldecanol, 2-undecylpentadecanol, oleyl alcohol or linoleyl alcohol.

The esters useful are esters of saturated or unsaturated, linear or branched C₁-C₂₆ aliphatic mono- or polyacids and of saturated or unsaturated, linear or branched C₁-C₂₆ aliphatic mono- or polyalcohols, the total carbon number of the esters being more particularly greater than or equal to 10.

Among the monoesters, mention may be made of dihydroabietyl behenate; octyldodecyl behenate; isocetyl behenate; cetyl lactate; C₁₂-C₁₅ alkyl lactate; isostearyl lactate; lauryl lactate; linoleyl lactate; oleyl lactate; (iso)stearyl octanoate; isocetyl octanoate; octyl octanoate; cetyl octanoate; decyl oleate; isocetyl isostearate; isocetyl laurate; isocetyl stearate; isodecyl octanoate; isodecyl oleate; isononyl isononanoate; isostearyl palmitate; methylacetyl ricinoleate; myristyl stearate; octyl isononanoate; 2-ethylhexyl isononate; octyl palmitate; octyl pelargonate; octyl stearate; octyldodecyl erucate; oleyl erucate; ethyl and isopropyl palmitates, 2-ethylhexyl palmitate, 2-octyldecyl palmitate, alkyl myristates such as isopropyl, butyl, cetyl, 2-octyldodecyl, myristyl or stearyl myristate, hexyl stearate, butyl stearate, isobutyl stearate; dioctyl malate, hexyl laurate, 2-hexyldecyl laurate.

Still in the context of this variant, esters of C₄-C₂₂ dicarboxylic or tricarboxylic acids and of C₁-C₂₂ alcohols and esters of mono-, di- or tricarboxylic acids and of C₂-C₂₆ di-, tri-, tetra- or pentahydroxy alcohols may also be used.

The following may especially be mentioned: diethyl sebacate; diisopropyl sebacate; diisopropyl adipate; di-n-propyl adipate; dioctyl adipate; diisostearyl adipate; dioctyl maleate; glyceryl undecylenate; octyldodecyl stearoyl stearate; pentaerythrityl monoricinoleate; pentaerythrityl tetraisononanoate; pentaerythrityl tetrapelargonate; pentaerythrityl tetraisostearate; pentaerythrityl tetraoctanoate; propylene glycol dicaprylate; propylene glycol dicaprate; tridecyl erucate; triisopropyl citrate; triisostearyl citrate; glyceryl trilactate; glyceryl trioctanoate; trioctyldodecyl citrate; trioleyl citrate; propylene glycol dioctanoate; neopentyl glycol diheptanoate; diethylene glycol diisononanoate; ethylene glycol distearate; diethylene glycol distearate and polyethylene glycol distearate.

The composition may also comprise, as fatty ester, sugar esters and diesters of C₆-C₃₀ and preferably C₁₂-C₂₂ fatty acids. It is recalled that the term “sugar” means oxygenous hydrocarbon-based compounds containing several alcohol functions, with or without aldehyde or ketone functions, and which comprise at least 4 carbon atoms. These sugars may be monosaccharides, oligosaccharides or polysaccharides.

Examples of suitable sugars that may be mentioned include sucrose (or saccharose), glucose, galactose, ribose, fucose, maltose, fructose, mannose, arabinose, xylose and lactose, and derivatives thereof, especially alkyl derivatives, such as methyl derivatives, for instance methylglucose.

The sugar esters of fatty acids may be chosen especially from the group comprising the esters or mixtures of esters of sugars described previously and of linear or branched, saturated or unsaturated C₆-C₃₀ and preferably C₁₂-C₂₂ fatty acids. If they are unsaturated, these compounds may comprise one to three conjugated or unconjugated carbon-carbon double bonds.

The esters according to this variant may also be chosen from mono-, di-, tri-, tetraesters and polyesters, and mixtures thereof.

These esters may be chosen, for example, from oleates, laurates, palmitates, myristates, behenates, cocoates, stearates, linoleates, linolenates, caprates and arachidonates, or mixtures thereof such as, especially, oleo-palmitate, oleo-stearate and palmito-stearate mixed esters.

It is more particularly preferred to use monoesters and diesters and especially sucrose, glucose or methyl glucose mono- or di-oleates, stearates, behenates, oleopalmitates, linoleates, linolenates and oleo stearates.

Advantageously, the composition of the present invention comprises from 1% to 35% by weight, preferably from 5% to 25% by weight, and more preferably from 1% to 20% by weight of the oil phase, relative to the total weight of the composition.

Unstable Active Agent

An active agent is one capable of bringing cosmetic effect to a user, especially to the skin, e.g., to face. Such cosmetic effect can be, for example, whitening, anti-aging, hydrating, and/or moisturizing. The active agents conventionally useful for a cosmetic product can be used in the composition according to the present invention, as long as it is oil soluble.

For the purpose of the present invention, the composition of the present invention is particularly applicable to use unstable active agents.

For the purpose of the present invention, by “unstable” we intend to mean an ingredient or composition undergoes significant change in its structure or properties within 2 months, or 1 month, in particular in solvent or solvated/dispersed state. Such change may be any one undesirable for the cosmetic use, including, but not limited to, color change, degradation, decomposition, reaction with others, significant evaporation, deposition, crystallization, and the like.

According to one embodiment of the invention, the unstable active agent is selected from the group consisting of retinoids, vitamin A, vitamin D, vitamin E, vitamin K, retinyl palmitate and phenylethyl resorcinol or a derivative thereof, such as ester or hydrophobic modification thereof.

Preferably, the unstable active agent is selected from the group consisting of group consisting of retinoids, wherein the retinoids are retinoic acid, retinol (vitamin A) and its esters, such as, retinyl propionate, and retinyl acetate, or retinyl palmitate.

More preferably, the unstable active agent is retinoid.

According to an embodiment, the composition of the present invention may comprise the unstable active agent in an amount ranging from 0.1% to 1% by weight, preferably from 0.2% to 0.5% by weight, relative to the total weight of the first composition.

Hydrophobic Particle (C)

The composition according to the present invention comprises hydrophobic particles. For the purpose of this invention, hydrophobic particles will adhere onto the surface of the oil phase, so as to reduce the oxidation of the unstable actives, e.g., retinoid in oil phase.

The particles may be mineral or organic, and may be in form of spherical particles or lamellar particles. The particles are preferably insoluble in the medium, e.g., solvent, oil phase, aqueous phase or the like, of the composition according to the invention, even at the melting point of the medium (e.g., about 100° C.).

In the present patent application, the term “spherical particles” means particles in the shape of sphere or at least approximate sphere, and/or a particle having an aspect ratio of about 0.9 to 1.1, e.g., about 0.95 to 1.05. For example, those skilled in the art can determine whether a particle is in the shape of sphere according to the conventional knowledge. In particular, regarding a given particle, it is within the ordinary skill of persons in the art to distinguish the shape of sphere from other shapes of plate, rod, powder, strip, irregular shapes or the like.

The term “lamellar particles” means herein particles in the shape of parallelepiped (rectangular or square surface), discoid shape (circular surface) or ellipsoid shape (oval surface), characterized by three dimensions: a length, a width and a height. In particular, regarding a given particle, it is within the ordinary skill of persons in the art to distinguish the lamellar shape from other shapes of sphere or the like.

According to the present invention, the hydrophobic particles may be selected from the group consisting of starches, talc, hydrophobic cellulose and derivatives thereof, hydrophobic silica, polyamide powders, hydrophobic pigments or any other hydrophobic particles which can be dispersed in aqueous phase.

The starches useful may be preferably modified, such as those derived, for example, from cereals such as wheat, corn or rice, from legumes such as white lentil, from tubers such as potato or cassava, tapioca starches; dextrins, such as corn dextrins; Amidon de Maïs B® from the company Roquette; potato feculent modified with 2-chloroethylaminodipropionic acid neutralized with sodium hydroxide, sold under the name Structure Solanace® by the company National Starch; native tapioca starch powder sold under the name Tapioca Pure® by the company National Starch.

The hydrophobic celluloses and derivatives thereof useful may be alkyl or hydroxyalkylcelluloses. Mention may be made especially of methylcelluloses, hydroxyalkylcelluloses, ethylhydroxyethylcelluloses and carboxymethylcelluloses. Examples that may be mentioned include the hydroxyethylcellulose sold under the name Natrosol™ 250 HHR PC by the company Ashland, or under the name Cellosize™ QP 4400 H by the company Amerchol (Dow Chemical), cetylhydroxyethylcelluloses sold under the names Polysurf 67CS® and Natrosol Plus 330® from Aqualon.

According to the present invention, the hydrophobic silica may be “hydrophobically modified” silica, which means one having subjected to lipophilic modification. According to the present invention, lipophilic modification means any organo-modification capable of bringing hydrophobic or lipophilic treatment to the silica. For example, a silica can be treated with a siloxane compound, e.g., hydroxydimethylsiloxane (or other silanes, such as silazane, etc.), through which the silanol group on the surface of the silica and the silanol group of the siloxane compound are condensed. Amongst others, useful examples of “lipophilically modified” silica can be made to silica silylate and silica dimethyl silylate, e.g., silica silylate, such as the one available from company TOKUYAMA CORPORATION under the name of AIRLICA® TL-10.

The composition of the present invention can comprise from 0.001% to 5% by weight, preferably from 0.02% to 2% by weight of the hydrophobic particles, relative to the total weight of the composition.

Antioxidant

The composition according to the present invention can further comprise an antioxidant.

The antioxidants used may include natural antioxidants such as phenols and carotenoids.

The antioxidant can include flavonoids. Flavonoids constitute a large class of more than 5,000 polyphenolic phytochemicals with antioxidant properties that act by direct free radical scavenging. Flavonoids have anti-inflammatory, antibacterial, antiviral, anti-allergic, anti-mutagenic, anti-thrombotic, anti-tumor and vasodilating effects and these methods of action can also be used to prevent, alleviate or eliminate oxidative damage from dental instruments. Flavonoids also exhibit chelation properties with metal ions and can mitigate oxidative damage from metal ions by chelating ions. The formation and stability of flavonoid-metal chelate is dependent on the function of the structure. Flavonoids having a catechol moiety and having a hydrogen bond between the hydroxyl groups at the 5-position and the 3-position have chelation properties.

Vitamin C and derivatives can be used, including ascorbic acid, erythorbic acid, or derivatives thereof, e.g., sodium ascorbate/erythorbate and the fat-soluble ester tetrahexyl decyl ascorbate/erythorbate and ascorbyl palmitate, magnesium ascorbyl phosphate, ascorbyl glucoside, glucosamine ascorbate, ascorbyl acetate, and the like. In addition, it is also possible to use plants derived from a large amount of vitamin C, such as extracts of Myrciaria dubia, acerola, Emblica officinalis, and bioflavonoids from rose hips and citrus, including water-soluble bioflavonoids such as hesperidin methyl chalcone.

Sesamum indicum or lignan may also be added. Sesame and its lignans (fibrous compounds associated with sesame) act as antioxidants. Sesame seed lignan significantly enhances vitamin E activity.

Other antioxidants which may be incorporated into the compositions of the present invention include tocopherols (e.g., d-alpha-tocopherol, d-beta-tocopherol, d-gamma-tocopherol, d-delta-tocopherol), tocotrienol Phenol (eg d-α-tocotrienol, d-β-tocotrienol, d-γ-tocotrienol, d-δ-tocotrienol) and vitamin E (α-tocopheryl acetate)). These compounds can be isolated from natural sources, prepared by synthetic means or mixed. The tocotrienol-rich vitamin E preparation can be obtained by fractionating the vitamin E preparation to remove a portion of the biophenol and recovering the higher concentrated tocotrienol product. Useful tocotrienols are natural products isolated, for example, from wheat germ oil, grain or palm oil using high performance liquid chromatography or from barley, distiller's grains or oats by alcohol extraction and/or molecular distillation. The term “tocotrienol” as used herein includes a tocotrienol-rich fraction obtained from these natural products as well as a pure compound. Increased glutathione peroxidase activity protects the skin from oxidative damage.

In addition, carotenoids, especially lutein types, are also useful antioxidants that can be used. Lutein-type carotenoids include molecules such as lutein, canthaxantin, cryptoxanthin, zeaxanthin and astaxanthin. Lutein compounds protect compounds such as vitamin A, vitamin E and other carotenoids.

The flavonoid may be a flavanone (a derivative of 2,3-dihydro-2-phenylbenzopyran-4-one). Flavanones include: scutellarin, eriodictin, hesperetin, hesperidin, sylvestre, isosakuranetin, naringenin, naringin, pinocin, tangrin (poncirin)), sakuranetin, sakura glycosides and 7-O-methyl ergophenol (Sterubin).

The flavonoid may be a dihydroflavonol (a derivative of 3-hydroxy-2,3-dihydro-2-phenylbenzopyran-4-one). Flavanols include: taxifolin, Aromadedrin, Chrysandroside A, Chrysandroside B, Xeractinol, astilbin, and flavonol.

The flavonoid may be a flavonoid (a derivative of 2-phenylbenzopyran-4-one). Flavonoids include: Apigenin, luteolin, tangeritin, Chrysin, baicalein, wild baicalein, wogonin, synthetic flavonoids: Diosmin and flavonoids ester.

The flavonoid may be a flavonol (a derivative of 3-hydroxy-2-phenylbenzopyran-4-one). Flavonols include: 3-hydroxyflavone, rhodoxanthin, quercetin, galangin, cotton dermatan, kaempferol, kaempferol, isorhamnetin, mulberry pigment, myricetin, naringin (Natsudaidain), Muskyl flavonol (Pachypodol), quercetin, methyl rhamnosin, rhamnetin, azalein, hyperoside, isoquercetin, kaempferol, myricetin, suede Glycosides, Robinin, Rutin, Spiraea, Xanthorhamnin, Amurensin, Icariin and Tracuridine.

The flavonoid may be a flavan-3-ol (a derivative of 2-phenyl-3,4-dihydro-2H-benzopyran-3-ol). Flavan-3-ol includes: catechin, epicatechin, epigallocatechin, epicatechin gallate, epigallocatechin gallate, epiafzelechin, Fisetinidol, Guibourtinidol, Mesquitol and Robinetinidol.

The flavonoid may be a flavan-4-ol (a derivative of 2-phenylchroman-4-ol). Flavan-4-ols include: Apiforol and Luteoforol.

The flavonoid may be an isoflavone (a derivative of 3-phenylbenzopyran-4-one). Isoflavones include: genistein, daidzein, garbanin A, formononetin, and equol metabolites from daidzein.

The antioxidant may be anthocyanin (a derivative of 2-phenylbenzopyranoside cation). Anthocyanins include: Aurantinidin, cyanidin, delphinidin, Europinidin, Luteolinidin, Pelargonidin, Malvidin, Peonyidin (Peonidin)), morning glory pigment (Petunidin), rose pigment (Rosinidin) and xanthone.

The antioxidant may be dihydrochalcone (a derivative of 1,3-diphenyl-1-propanone). Dihydrochalcone includes: phloretin, dihydrochalcone phloridin cisplatin, Aspalathin, naringin dihydrochalcone, neohesperidin dihydrochalcone and Nothofagin. The mode of action of the present invention is not limited, but dihydrochalcone can exert an antioxidant effect by reducing active radicals such as active oxygen and reactive nitrogen species.

The antioxidant can be anthocyanin. Anthocyanins and their derivatives are antioxidants. Anthocyanins comprise a class of flavonoid compounds responsible for the red, purple and blue colors of many fruits, vegetables, grains and flowers, which are naturally occurring water-soluble compounds. In addition, anthocyanins are collagenase inhibitors. Inhibition of collagenase helps prevent and reduce wrinkles caused by skin collagen reduction, increase skin elasticity, and the like. Anthocyanins can be obtained from any part of a variety of plant sources, such as solids, flowers, stems, leaves, roots, bark or seeds. Those skilled in the art will appreciate that certain portions of the plant may contain higher natural levels of anthocyanins, and thus these moieties are used to obtain the desired anthocyanins. In some cases, the antioxidant can include one or more betaine. Betatin, similar to anthocyanins, is available from natural sources and is an antioxidant.

The antioxidant may be a phenylpropanoid (a derivative of cinnamic acid). Phenylpropanoids include: cinnamic acid, caffeic acid, ferulic acid, trans-ferulic acid (including its antioxidant pharmacore 2,6-dihydroxy acetophenome), 5-hydroxyferic acid, sinapic acid, Coumarin, coniferyl alcohol, sinapyl alcohol, eugenol, Chavicol, baicalein, P-coumaric acid and sinapinic acid. Without limiting the mode of action of the present invention, phenylpropanoids can neutralize free radicals.

The antioxidant may be chalcone (a derivative of 1,3-diphenyl-2-propen-1-one). Chalcone includes: zirconia, Okanin, safflower, Marein, Sophoradin, Xanthohumol, Flavokvain A, Flavokavain B, Flavokavin C and Synthetic Safalcone.

The antioxidant may be curcuminoid. Curcuminoids include: curcumin, demethoxycurcumin, bis-demethoxycurcumin, tetrahydrocurcumin, and tetrahydrocurcumin. Curcumin and tetrahydrocurcumin can be derived from the rhizome of turmeric. Tetrahydrocurcumin, a metabolite of curcumin, has been found to be a more potent antioxidant and more stable than curcumin.

The antioxidant can be tannin. Tannins include: tannins, Terflavin B, Glucogallin, Dgallic acid, and Quercitannic acid.

The antioxidant can be a stilbenoid. The mites include: resveratrol, red sandalwood and paclitaxel. Resveratrol can include, but is not limited to, 3,5,4′-trihydroxyindole, 3,4,3′,5′-tetrahydroxyindole (cetotriol), 2,3′,4,5′-Tetrahydroxyindole (oxidized resveratrol), 4,4′-dihydroxyindole and its alpha and beta glucoside, galactoside and mannoside derivatives.

The antioxidant may be coumarin (a derivative of 2H-benzopyran-2-one). Coumarins include: 4-hydroxycoumarin, umbelliferone, Aesculetin, Herniarin, Auraptene, and dicoumarin.

The antioxidant can be a carotenoid. Carotenoids include: beta-carotene, alpha-carotene, gamma-carotene, beta-cryptoxanthin, lycopene, lutein and idebenone.

The antioxidant may be: xanthone, butylated hydroxytoluene, 2,6-di-tert-butylphenol, 2,4-dimethyl-6-tert-butylphenol, gallic acid, eugenol, uric acid, α-lipoic acid, ellagic acid, cichoric acid, chlorogenic acid, rosmarinic acid, salicylic acid, acetylcysteine, S-allylcysteine, pyridone (Barbigerone), Chebulagic acid, edaravone, ethoxyquin, glutathione, hydroxytyrosol, idebenone, melatonin, N-acetyl serotonin, nordihydroguaiac Acid, Oleotanthal, oleuropein, Paradol, paclitaxel, probucol, propyl gallate, protocatechuic acid, pyrithione, rutin, flax lignan diglucoside, sesamin, sesame phenol, Silybin, silymarin, theaflavins, theaflavins digallate, Thmoquinone, Trolox, tyrosol, polyunsaturated fatty acids and sulfur-based antioxidants such as methionine or lipoic acid.

The antioxidants can be synthetic antioxidants, such as butylated hydroxy toluene (BHT), butylated hydroxy anisole (BHA), tert-butyl hydroquinone (TBHQ) and phospholipid.

Other useful antioxidants may comprise water soluble antioxidants, such as tea polyphenols, or oil soluble antioxidants, such as pentaerythrityl tetra-di-t-butyl hydroxyhydrocinnamate (Tinogard TT), diethylhexyl syringylidenemalonate, etc.

Without being limited by any known theory, it is believed that the oxidation of retinol or retinol derivatives can be avoided/reduced through the reaction of antioxidants with oxygen.

According to the present invention, the antioxidant can be present in either the aqueous phase or the oil phase, or the both. Preferably, the antioxidant can be present in the oil phase.

The antioxidant is preferably used according to the invention in an amount which may range from 0.2% to 10% by weight, preferably from 0.5% to 10% by weight both in oil phase as well as in aqueous phase, and more preferably from 1% to 5% by weight, relative to the total weight of the composition.

Process to Form the Composition

The process to form the composition is not limited specifically, as long as the unstable active agent can be distributed uniformly in the oil phase.

According to the present invention, the hydrophobic particles (C) are added into alcohol, e.g., ethanol to obtain a mixture 1. Then, mixture 1 is added into the aqueous phase (A) to obtain a mixture 2. Subsequently, mixture 2 is added into the oil phase (B) to obtain the composition according to the present invention. During the additions, stirring or any other means known in the art facilitating the homogeneous mixing may be applied.

An example for the process comprises the steps of:
1). Adding the hydrophobic particles (C) into alcohol, with agitating till particle dispersed, to obtain mixture 1.
2). Mixing the materials for the aqueous phase (A) until all materials dissolved, to obtain the aqueous phase (A).
3). Adding mixture 1 into phase (A), with stirring till uniform, to obtain mixture 2.
4). Mixing the materials for the oil phase (B), with stirring till all materials dissolved, to obtain the oil phase (B).
5). Adding mixture 2 into the oil phase (B), with stirring for 30-40 seconds.

Following the process according to the present invention, in the composition obtained, the oil phase can be dispersed in water or under water. Accordingly, without being limited by any known theory, it is believed that during storage, the oil phase is isolated from the environment by the aqueous phase.

Package

Corresponding to the composition according to the present invention, the invention further provides a package comprising:
1) a container; and
2) the bi-phase composition according to the present invention within the container;
wherein the aqueous phase (A) and the oil phase (B) is placed in such a way that when the container is opened, the oil phase (B) is separate from environment by the aqueous phase (A).

Accordingly, for the package, the oil phage (B) can be placed far away from the opening of the container, being separated with the aqueous phase (A). According to an embodiment, the oil phase (B) is dispersed within the aqueous phase (A).

According to an embodiment, the oil phase (B) is placed under the aqueous phase (A), and the container has an opening on the top, in which the said “top” is assigned regarding the conventionally vertical direction to store the container. In this case, for example, the user can shake the container to mix the phases well before application.

According to another embodiment, the oil phase (B) is placed on the aqueous phase (A), and the container has an opening on the bottom, in which the said “bottom” is assigned regarding the conventionally vertical direction to store the container. Here, for example, when a pump is used for the container, the user can vertically reverse the container, shake the container to mix the phases well, and take out the composition.

### EXAMPLES

The ingredient amounts/concentrations in the compositions/formulas described below are expressed in % by weight, relative to the total weight of each composition/formula.

### I. Preparation

The compositions of inventive examples 1-3 and comparative example 1 were prepared as follows.

TABLE 1

| Components | | Ex. 1 | Ex. 2 | Ex. 3 | CE. 1 |
|---|---|---|---|---|---|
| Phase | WATER | QS | QS | QS | QS |
| A | GELLAN GUM | 0.085 | 0.85 | 0 | 0 |
| | XANTHAN GUM | 0.085 | 0.85 | 0 | 0 |
| | ASCORBIC ACID | 0.1 | 0.1 | 0.1 | 0.1 |
| | TRISODIUM | 0.2 | 0.2 | 0.2 | 0.2 |
| | ETHYLENEDIAMINE | | | | |
| | DISUCCINATE | | | | |
| | CALCIUM CHLORIDE | 0.5 | 0.5 | 0 | 0 |
| Phase | ALCOHOL | 5 | 5 | 5 | 5 |
| B | ETHYLCELLULOSE | 0.003 | 0 | 0 | 0 |
| | Silica Dimethyl Silylate | 0 | 0.1 | 0.1 | 0 |
| Phase | TOCOPHEROL | 0.1 | 0.1 | 0.1 | 0.1 |
| C | ACETYL TRIBUTYL | 3 | 3 | 4 | 3 |
| | CITRATE | | | | |
| | RETINOL | 0.2 | 0.2 | 0.2 | 0.2 |
| | PENTAERYTHRITYL | 0.3 | 0.3 | 0.3 | 0.3 |
| | TETRA-DI-T-BUTYL | | | | |
| | HYDROXY- | | | | |
| | HYDROCINNAMATE | | | | |
| | DIETHYLHEXYL | 2 | 2 | 2 | 2 |
| | SYRINGYLIDENE- | | | | |
| | MALONATE | | | | |
| | DIETHYLHEXYL | 0.45 | 0.45 | 0.45 | 0.45 |
| | SYRINGYLIDENE- | | | | |
| | MALONATE | | | | |

Protocol of preparation using the third composition in table 1:
1). Mixing the materials of phase B, with stirring till particle dissolved;
2). Adding all materials in a container 1 for phase A, with stirring until all materials dissolved;
3). Adding phase B to the container 1, with stirring till uniform;
4). Adding all materials in another container 2 for phase C, with stirring till all materials dissolved; and
5). Adding phase C into the container 1, with stirring for 30 seconds.

### II. Evaluation of the Inventive Compositions and Comparative Examples

The inventive compositions and comparative examples were evaluated using the following protocols.

Method for Texture Transformation Evaluation

Retinol contents of all samples from the inventive compositions and comparative example over time were evaluated via HPLC system for TO (immediately after preparation) and T2 month (two months after preparation). Specifically, after preparation, all the samples were divided into two equal parts, for which the first half parts were tested for retinol contents at TO, while the second haft parts were stored under 45° C. for two months. At T1, the second haft parts were tested for the retinol contents.

For the test, all the samples were pretreated via centrifugation to separate the aqueous phase and the oil phase. Then oil phase was directly tested in HPLC system. After the HPLC analyze, the accurate the data of retinol content was finally got, as showed in Table 2 below.

TABLE 2

| Properties | Ex. 1 | Ex. 2 | Ex. 3 | CE. 1 |
|---|---|---|---|---|
| Retinol Content | 0.2 | 0.2 | 0.2 | 0.2 |
| (T0) | | | | |
| Retinol Content | 0.19 | 0.18 | 0.19 | 0.11 |
| (Under 45° C. two month) | | | | |
| Retinol Loss compared to T0 | 5% | 10% | 5% | 45% |

Example 1 or 2 was suspending system. Oil phase was suspended in oil droplets. The retinol protecting system was supported by pickering structure and the antioxidant. The example 3 was another format (oil phase under water phase) with similar retinol protecting system. All of examples 1-3 showed excellent retinol stability. Less than 10% by weight retinol was lost after 2 months under 45° C. condition. As compared, CE.1 was an oil-on-water system. In this formula, only the antioxidant was involved, without any pickering structure. As compared, 45% by weight retinol was lost after 2 months under 45° C.

The foregoing description illustrates and describes the present disclosure. Additionally, the disclosure shows and describes only the preferred embodiments of the disclosure, but, as mentioned above, it is to be understood that it is capable of changes or modifications within the scope of the concept as expressed herein, commensurate with the above teachings and/or skill or knowledge of the relevant art. The described hereinabove are further intended to explain best modes known of practicing the disclosure and to enable others skilled in the art to utilize the disclosure in such, or other embodiments and with the various modifications required by the particular applications or uses disclosed herein. Accordingly, the description is not intended to limit the disclosure to the form disclosed herein. Also it is intended that the appended claims be construed to include alternative embodiments.

All publications, patents and patent applications cited in this specification are herein incorporated by reference, and for any and all purposes, as if each individual publication, patent or patent application were specifically and individually indicates to be incorporated by reference. In this case of inconsistencies, the present disclosure will prevail.

1. A bi-phase composition comprising:
(A) a continuous aqueous phase;
(B) an oil phase comprising an unstable active agent, and
(C) hydrophobic particles adhered on the surface of the oil phase.

2. The composition according to claim 1, further comprising an antioxidant, either in the aqueous phase (A) and/or in the oil phase (B).

3. The composition according to claim 1, wherein the aqueous phase (A) comprises water in an amount ranging from 65% to 99% by weight relative to the total weight of the composition.

4. The composition according to claim 1, wherein the oil phase (B) comprises a substance selected from the group consisting of lower alkanes, fatty alcohols, fatty acid esters, fatty alcohol esters, oils, non-silicone waxes, and silicone oils.

5. The composition according to claim 1, wherein the oil phase (B) is present in the composition from 5% to 25% by weight relative to the total weight of the composition.

6. The composition according to claim 1, wherein the unstable active agent is selected from the group consisting of retinoids, vitamin B group, vitamin D, vitamin K, nicotinic acid, folic acid, pantothenic acid, ferulic acid, ascorbic acid 2-glucoside, dipalmiroyl hydroxyproline, and a salt or ester thereof.

7. The composition according to claim 1, wherein the unstable active agent is selected from the group consisting of retinoic acid, retinol (vitamin A) and its esters.

8. The composition according to claim 1, wherein the unstable active agent is retinol, retinyl acetate, or retinyl palmitate.

9. The composition according to claim 1, wherein the hydrophobic particles (C) are in form of spherical particles or lamellar particles.

10. The composition according to claim 1, wherein the hydrophobic particles (C) are selected from the group consisting of starches, talc, hydrophobic cellulose and derivatives thereof, hydrophobic silica, polyamide powders, and hydrophobic pigments.

11. The composition according to claim 1, wherein the hydrophobic particles (C) are present in an amount of from 0.001% to 5% by weight relative to the total weight of the composition.

12. The composition according to claim 1, further comprising an antioxidant selected from the group consisting of a natural exogenous phytochemical antioxidant, a flavonoid, a vitamin, tocopherols, tocotrienol phenol, carotenoid, anthocyanin, dihydrochalcone, phenylpropanoid, chalcone, curcuminoid, tannin, stilbenoid, coumarin, carotenoid, butylated hydroxy toluene (BHT), butylated hydroxy anisole (BHA), tert-butyl hydroquinone (TBHQ), tetra-di-t-butyl hydroxyhydrocinnamate (Tinogard TT), diethylhexyl syringylidenemalonate and a mixture thereof.

13. The composition according to claim 13, wherein the antioxidant is present in an amount of from 0.2% to 10% by weight relative to the total weight of the composition.

14. A package comprising:
1) a container; and
2) the bi-phase composition according to claim 1 within the container;
wherein the aqueous phase (A) and the oil phase (B) is placed in such a way that when the container is opened, the oil phase (B) is separate from environment by the aqueous phase (A).

15. The package according to claim 14, wherein the oil phase (B) is placed under the aqueous phase (A), or the oil phase (B) is dispersed within the aqueous phase (A).

16. The composition according to claim 1, wherein the oil phase (B) comprises an oils which is a mineral, plant, animal or synthetic non-silicone oil.

17. The composition according to claim 1, wherein the unstable active agent is selected from the vitamin B group and is selected from the group consisting of vitamin B5 (panthenol), vitamin B3 (niacinamide), vitamin B1 and vitamin B2.

18. The composition according to claim 1, wherein the unstable active agent is a retinol ester and is selected from the group consisting of retinyl propionate, retinyl acetate and retinyl palmitate.
